(19) United States
(12) Patent Application Publication
(10) Pub. No.: US 2025/0170290 A1
(43) Pub. Date: May 29, 2025

(54) METHOD OF PREPARING A CLEANING UNIT BY A PREPARATION STATION, METHOD OF CLEANING A HOUSING OF A MEDICAL IMAGING DEVICE, AND PREPARATION STATION

(71) Applicant: Hans Georg HAGLEITNER, Zell am See (AT)

(72) Inventor: Hans Georg HAGLEITNER, Zell am See (AT)

(21) Appl. No.: 18/958,161

(22) Filed: Nov. 25, 2024

(30) Foreign Application Priority Data
Nov. 24, 2023 (AT) A 50950/2023

(51) Int. Cl. A61L 2/00

(52) U.S. Cl. CPC A61L 2/16; A61L 2/26; A61L 2202/24

(57) ABSTRACT

A method for preparing a cleaning unit that is provided for cleaning a housing of a medical imaging device that surrounds a patient-accommodating region. The cleaning unit has a support structure and a cleaning component, and it is prepared by a preparation station that is separate from the medical imaging device and arranged in the vicinity of the same. The cleaning unit is moved toward the preparation station and at least one preparation step is subsequently carried out in order to prepare the cleaning unit for subsequent cleaning.

### CROSS-REFERENCE TO RELATED APPLICATION

This application claims the priority, under 35 U.S.C. § 119, of Austrian Patent Application A 50950/2023, filed Nov. 24, 2023; the prior application is herewith incorporated by reference in its entirety.

### FIELD AND BACKGROUND OF THE INVENTION

The present invention relates to a method for preparing a cleaning unit, the cleaning unit being provided to clean a housing of a medical imaging device that surrounds a patient-accommodating region, to a method for cleaning a housing of a medical imaging device that surrounds a patient-accommodating region, a preparation station for a method for preparing and/or cleaning such a cleaning unit, to such a preparation station, and to an arrangement with such a preparation station.

Medical imaging devices are used in radiology and include MRI (magnetic resonance imaging) and CT (computed tomography) equipment, for example. These are dirtied during use and/or contaminated with germs and/or viruses. For example, within the medical imaging device, this can be caused by the persons to be examined themselves, should germs or other contamination be left behind following coughing, sneezing, humid respiration, physical contact with the medical imaging device or the like. Such contamination remains, inter alia, on the inner side of the medical imaging device, specifically on a housing.

In this case, manual cleaning is brought about using a cleaning utensil that is optionally secured to a rod, for example as disclosed in U.S. Pat. No. 5,918,342.

A disadvantage thereof is that that type of cleaning is very laborious and time consuming, and/or the cleaning staff is located very close or sometimes even within the medical imaging device. The radiological influence by the medical imaging device, for example x-ray radiation or magnetic fields, can be uncomfortable in that context.

The provision of automated cleaning units within the medical imaging device is also known, for example as disclosed in Chinese utility model publication CN 219251060 U or German published patent application DE 10 2021 210 471 A1. In that case, the cleaning unit can perform wiping-type cleaning, by virtue of the cleaning unit being displaced from one end of the housing to the other end of the housing.

A disadvantage thereof is that the preparation of such a cleaning unit is time intensive and complicated. The medical imaging device cannot be used or used only to a limited extent during the preparation, whereby the operating time of the medical imaging device is restricted.

### SUMMARY OF THE INVENTION

The problem to be solved by the present invention thus consists in rectifying the disadvantages of the prior art at least in part, and in specifying an improved method over the prior art for preparing a cleaning unit, wherein the method is distinguished by less outlay for the preparation staff in particular. Further, the problem consists in specifying a method for cleaning a cleaning unit, a preparation station for a method for preparing and/or cleaning such a cleaning unit and an arrangement having such a preparation station.

With the above and other objects in view there is provided, in accordance with the invention, a method for preparing a cleaning unit that is configured for cleaning a housing of a medical imaging device surrounding a patient-accommodating region, the cleaning unit having a support structure and a cleaning component, the method comprising:
providing a preparation station separate from the medical imaging device and arranged in a vicinity of the medical imaging device;
moving the cleaning unit towards the preparation station; and
subsequently carrying out at least one preparation step for preparing the cleaning unit for a subsequent cleaning.

In other words, the above and other objects are achieved, in accordance with the invention, by a method for preparing a cleaning unit, wherein the cleaning unit being provided to clean a housing of a medical imaging device that surrounds a patient-accommodating region and the cleaning unit comprising a support structure and a cleaning component, by means of a preparation station that is separate from the medical imaging device and arranged in the vicinity of same; wherein:
the cleaning unit is moved toward the preparation station and
at least one preparation step is subsequently carried out, by means of which the cleaning unit is prepared for subsequent cleaning.

Different preparation steps are often needed for the preparation of a cleaning unit. If a cleaning unit is prepared for a new imaging process directly on the medical imaging device, then this at least increases the time expenditure before the new imaging process can be carried out. For example, a patient must await the completion of cleaning unit preparations before they can take their place on a patient table, also referred to as a scan table, scanner table, or scanner bed. The method according to the invention enables preparation of the cleaning unit by means of a preparation station that is separate from the medical imaging device and arranged in the vicinity of same, in such a way that the medical imaging device is at least partly accessible and at least partly usable in the meantime. For example, the method for preparing the cleaning unit can take place during a patient changeover. A patient changeover requires time, especially in the case of elderly and frail patients, and the cleaning unit can be prepared at the same time; hence, less time expenditure is achieved because the cleaning unit is prepared at the preparation station and the patient-accommodating region is more easily accessible.

An advantage of such a method for preparing the cleaning unit is that at least one preparation step for preparing the cleaning unit can be implemented by means of the preparation station. The preparation station thus facilitates at least one preparation step, preferably at least one completely manual preparation step, and thus reduces the preparation outlay for the preparation staff.

In the instant context, the term “cleaning” is understood to mean cleansing, disinfecting and/or sterilizing. In this case, cleaning can be implemented in the form of wiping, abrading, scraping, rubbing, scrubbing, washing and/or the like.

The expression “preparation station arranged in the vicinity of same” is understood to mean that the preparation station is close to the medical imaging device, with the distance between the preparation station and the medical imaging device being sufficient to ensure that the radiological influence, for example of x-ray radiation or a magnetic field of the medical imaging device, does not adversely affect the preparation station, in particular metallic components and/or electronic components of the preparation station, and/or the preparation staff when carrying out a method for preparing the cleaning unit. For example, a sufficient distance between the preparation station and the medical imaging device can be at least 0.5 meters.

The term “preparation steps” means all measures that are performed on a cleaning unit before, during and/or after a medical imaging process.

Provision can be made for the cleaning unit to be parked in the preparation station before and/or after the at least one preparation step.

For the sake of completeness, attention is drawn to the fact that the numerals used within the description of this invention, for instance one, two, three and the like, as a matter of principle only describe the minimum quantity present of a feature of the methods according to the invention, of the preparation station according to the invention and/or of the arrangement according to the invention having such a preparation station. It is self-evident that individual features or constituent parts can also be present in a greater quantity. For example, methods according to the invention might include more than one preparation step, and a preparation station according to the invention might comprise more than one preparation device, etc. In this sense, the numeral one, for example, should thus advantageously be understood in the sense of at least one, etc.

In a preferred embodiment variant, provision can be made for the method for preparing a cleaning unit and/or the method for cleaning a housing of a medical imaging device that surrounds a patient-accommodating region to comprise at least one or at least two or at least three or at least four or at least five preparation steps.

For example, at least three preparation steps might be provided, wherein the deformation of the cleaning component, the placement of the cleaning covering and the application and/or introduction of a cleaning product can each be a preparation step.

In another example, a fourth preparation step might be provided in addition to the previous example, wherein the cleaning product is applied and/or introduced a second time or another cleaning product is additionally applied and/or introduced.

The individual preparation steps disclosed here can be repeated and/or combined with one another within the scope of the method for preparing a cleaning unit and/or the method for cleaning a housing of a medical imaging device that surrounds a patient-accommodating region.

In a preferred embodiment variant, provision can be made for the preparation station to comprise at least one or at least two or at least three or at least four or at least five preparation devices.

For example, at least three preparation devices might be provided, wherein the deformation device, the placement device and the application device might each be a preparation device.

In another example, a fourth preparation device might be provided in addition to the previous example, wherein additional provision might be made for a second application device for a second application and/or introduction of the cleaning product or for an application of a different cleaning product.

The individual preparation devices disclosed here can be provided multiple times and/or combined with one another in the preparation station for preparing a cleaning unit and/or for cleaning a housing of a medical imaging device that surrounds a patient-accommodating region.

In a further preferred embodiment variant of the invention, the method may be at least partly automated, or even more preferably completely automated.

A preferred embodiment of the method provides for at least one of the following method steps to be provided:
detachably connecting the cleaning unit to the preparation station, preferably by means of a coupling device,
charging a force accumulator and/or an energy accumulator,
deforming, preferably inflating, the cleaning component by filling with a pressure medium or by removing the pressure medium,
placing, preferably stretching, a cleaning covering of the cleaning component on a cleaning main body of the cleaning component, preferably along an outer contour of the cleaning main body,
applying and/or introducing a cleaning product onto and/or into a cleaning covering of the cleaning component, preferably before, during and/or after placement of the cleaning covering of the cleaning component on a cleaning main body of the cleaning component.

Preparation steps, for example a deformation of the cleaning component, a placement of the cleaning covering and/or an application and/or introduction of a cleaning product can be time consuming and/or complicated, which is why the use of a preparation station reduces the dead time, specifically the unused time, within a method for preparing a cleaning unit and/or uses the operating time of a medical imaging device better and more efficiently.

In the simplest case, a detachable connection can be present between the cleaning unit and the preparation station if the cleaning unit is placed onto or into the preparation station. Instead of that or in addition, provision can be made for the provision of a dedicated coupling device for the detachable connection.

The terms “detachable connection”, “detachably connected” and “detachably connectable” can be understood to mean that at least two initially separate components can be connected to one another, wherein the detachable connection can subsequently be non-destructively undone, with the result that the at least two components are yet again present in their original separated state, in order to be able to be subsequently connected again. “Detachable connection”, “detachably connected” and “detachably connectable” can therefore also be referred to as “non-destructively detachable connection”, “non-destructively detachably connected” and “non-destructively detachably connectable.”

An energy accumulator serves to store currently available but non-required energy for later use. An energy accumulator can be an electrical energy accumulator such as a battery, for example. A force accumulator serves analogously for the provision of force for later use. A force accumulator can be a mechanical force accumulator such as a spring device, for example.

At least one preparation step for preparing the cleaning unit and/or a cleaning step for cleaning a housing that surrounds a patient-accommodating region can be carried out at a later time with the aid of an energy accumulator or force accumulator. For example, a spring as force accumulator can be charged during the preparation in order to cause a deformation of the cleaning component at a later stage, preferably when the cleaning unit is arranged within the housing during the cleaning operation. In this way, the cleaning component of the cleaning unit can be pressed against the housing within the housing, or the pressure can be terminated, by way of the charged spring.

In the context, the terms “deformation” and/or “deformable” are understood to mean that the shape of a body can be modified as a consequence of the action of a force and/or a stress. A deformation of a body can be decomposed into a first component, in which the volume and/or the spatial extension of the body changes, but not the geometric shape of the body, for example in the event of an increase in the radius of a sphere or a tubular body, and into a second component, in which the geometric shape of the body changes, but not the volume and/or the spatial extension of the body, for example in the event of a sphere being stretched along one axis to form an ellipsoid. In the event of a deformation as a result of the cleaning component being filled with a pressure medium, there can be a deformation with a first and/or a second component.

Deformability of the cleaning component as a result of filling with a pressure medium causes a modification of a geometric shape, preferably an external diameter, of the spatial extension and/or the volume of the cleaning component. Contact between the cleaning unit arranged in the medical imaging device and the housing of the medical imaging device can be brought about or omitted in a targeted manner in this way.

Consequently, reliable cleaning of a housing can be performed by virtue of the cleaning component being put into a filled state, the housing being contacted by the filled cleaning component and the cleaning component cleaning the housing, for example by wiping, when the cleaning unit is moved.

A “non-filled cleaning component” or a “cleaning component in a non-filled state” is a cleaning component which is not filled with a pressure medium, wherein the non-filled cleaning component of a cleaning unit inserted into the housing does not come into contact with the housing.

In a non-filled state, the cleaning component is affected by force and/or stress due to the atmospheric pressure surrounding the cleaning component and optionally due to a medium, not the pressure medium, present in the cleaning component, but the said cleaning component is by no means affected by pressure from the housing.

A “partly filled cleaning component” or a “cleaning component in a partly filled state” is a cleaning component which is filled at least in part with a pressure medium, wherein the partly filled cleaning component of a cleaning unit inserted into the housing does not come into contact with the housing.

A “filled cleaning component” or a “cleaning component in a filled state” is a cleaning component which is filled with a pressure medium, wherein the filled cleaning component of a cleaning unit inserted into the housing comes into contact with the housing.

In a filled state, the cleaning component is affected by force and/or stress due to the pressure medium, due to the atmospheric pressure surrounding the cleaning component and due to the housing in contact with the cleaning component and optionally by a reactive force of the cleaning component, the preferably elastic cleaning component, as a consequence of the deformation.

In the transition from a filled cleaning component, in which there is contact with the housing, via a partly filled cleaning component to a non-filled cleaning component, pressure medium is removed from the cleaning component, whereby a spatial extension of the cleaning component is reduced, and contact with the housing is terminated.

In the transition from a non-filled cleaning component, in which there is no contact with the housing, via a partly filled cleaning component to a filled cleaning component, pressure medium is introduced into the cleaning component, whereby a spatial extension of the cleaning component is increased, and contact with the housing is achieved.

Only after a sufficient amount of pressure medium has been introduced into a cleaning component of a cleaning unit or, in other words, only after the cleaning component has been sufficiently filled with pressure medium does the transition occur from the partly filled cleaning component to the filled cleaning component, whereby contact with the housing by way of a cleaning unit inserted into the housing is made possible. Should an already filled cleaning component be subsequently filled even more with pressure medium, there is pressurized contact with the housing, wherein the contact pressure is determined by the degree of filling of the filled cleaning component.

A cleaning component can be filled a plurality of times in succession and increase its spatial extension according to the explanations given above every time it is refilled. This applies analogously to a cleaning component from which some of the pressure medium is taken a plurality of times in succession.

Here, a “cleaning product” can be understood to mean a well-known product for cleaning purposes, for example soap, a disinfectant, for example alcohol, and/or a sterilization means.

A preferred embodiment of the method provides for at least one of the following method steps to be provided:
detaching the cleaning unit detachably connected to the preparation station, preferably by means of the coupling device,
transferring the cleaning unit between the medical imaging device and the preparation station,
detachably connecting the cleaning unit to the medical imaging device,
detaching the cleaning unit from the medical imaging device.

In a preferred embodiment variant, provision can be made for the transfer of the cleaning unit to be implemented by means of a transfer device.

Furthermore, protection is sought for a method for cleaning a housing of a medical imaging device that surrounds a patient-accommodating region
by means of a cleaning unit, the cleaning unit comprising a support structure and a cleaning component, and
by means of a preparation station, preferably separate from the medical imaging device and arranged in the vicinity of same, having at least one preparation device and having at least one displacement device, the cleaning unit being detachably connected to the preparation station,
   wherein at least one preparation step is carried out by the at least one preparation device, by means of which the cleaning unit is prepared for subsequent cleaning, and
   wherein cleaning is implemented by moving the prepared cleaning unit within the medical imaging device in at least one direction, preferably in at least two directions.

In a preferred embodiment variant of the method for cleaning a housing of a medical imaging device that surrounds a patient-accommodating region, provision can be made for the at least one displacement device to be part of the preparation station or for the at least one displacement device to be part of the medical imaging device, for example a scanner table.

A less complex and more efficient workflow can be achieved by means of a preparation station, by virtue of the preparation station firstly facilitating the preparation of a cleaning unit and secondly being able to be used as displacement device for cleaning the housing.

In a preferred embodiment variant, completion of the movement of the prepared cleaning unit along the housing in a first direction can be immediately followed by converting the filled cleaning component into a non-filled state and the cleaning unit with the non-filled cleaning component being moved contactlessly back into the initial position. In the process, there is no renewed contact between the cleaning component and housing, whereby recontamination is avoided.

With the above and other objects in view there is also provided, in accordance with the invention, a preparation station for preparing a cleaning unit, the cleaning unit being provided to clean a housing of a medical imaging device that surrounds a patient-accommodating region and the cleaning unit comprising a support structure and a cleaning component, wherein the preparation station is separate from the medical imaging device and arranged in the vicinity of same, wherein the preparation station comprises at least one preparation device, by means of which the cleaning unit is prepared for subsequent cleaning.

A preferred embodiment of the preparation station provides for the at least one preparation device to be:
a coupling device for detachably connecting the cleaning unit to the preparation station;
a charging device for charging a force accumulator and/or an energy accumulator;
a deformation device for deforming the cleaning component;
a placement device for placing a cleaning covering of the cleaning component on a cleaning main body of the cleaning component; or
an application device for applying a cleaning product onto and/or into a cleaning covering of the cleaning component.

In a preferred embodiment variant, provision can be made for the preparation station to be detachably connected or connectable to the cleaning unit.

In a preferred embodiment variant, provision can be made for at least one of the preparation devices to be at least partly automated, preferably be completely automated.

A preferred embodiment variant can provide for the provision of at least one open-loop and/or closed-loop control unit for open-loop and/or closed-loop control of at least one of the preparation devices.

For example, an open-loop and/or closed-loop control unit can be used for open-loop and/or closed-loop control of the amount of pressure medium and/or the pressure to be attained within the filled cleaning component.

A preferred embodiment variant can provide for the provision of at least one sensor, preferably wherein the sensor is connected to at least one open-loop and/or closed-loop control unit.

A sensor, preferably a pressure sensor, can be used to monitor the state of the filled and/or non-filled cleaning component.

The efficiency of cleaning the housing with the cleaning unit can be increased, the efficiency of the operation of the medical imaging device can be raised and the preparation outlay for preparation staff can be reduced by the use of at least partly automated, preferably completely automated, preparation devices such as a deformation device, a placement device and/or an application device, for example with the aid of an open-loop and/or closed-loop control unit and/or with the aid of sensors.

According to a preferred embodiment of the preparation station, provision is made for the deformation device to comprise a deformation drive, a pressure medium and a connecting line for connecting, preferably detachably or non-detachably connecting, the cleaning component to the deformation drive, wherein the pressure medium can be filled into the cleaning component and/or removed from the cleaning component by means of the deformation drive, wherein the cleaning component is deformable by filling with the pressure medium and/or removing the pressure medium.

As noted above, the terms “detachable connection”, “detachably connected” and “detachably connectable” may be understood as “non-destructively detachable connection”, “non-destructively detachably connected” and “non-destructively detachably connectable.”

Similarly, the terms “non-detachable connection,” “non-detachably connected” and “non-detachably connectable” can be understood to mean that at least two initially separate components can be connected to one another, wherein this non-detachable connection cannot be subsequently released without the integrity of at least one of the at least two components being damaged and/or without a renewed connection of the at least two components being made more difficult or being precluded. “Non-detachable connection”, “non-detachably connected” and “non-detachably connectable” can therefore also be referred to as “non-destructively non-detachable connection”, “non-destructively non-detachably connected” and “non-destructively non-detachably connectable.”

In a preferred embodiment variant, provision can be made for a deformation device to comprise a compressor, preferably an air compressor, wherein the compressor aspirates the pressure medium, preferably ambient air, guides the latter under pressure to the cleaning component via a connecting line and thus fills the cleaning component.

According to a preferred embodiment of the preparation station, provision is made for the cleaning covering to be able to be placed, preferably stretched, on the cleaning main body, preferably along an outer contour of the cleaning main body, by means of the placement device.

The placement device can be used to remove a cleaning covering from the cleaning unit or, in other words, displace a cleaning covering of the cleaning unit. Hence, a preferred embodiment variant can include a removal of a first, preferably used cleaning covering and/or a placement of a second, preferably unused cleaning covering.

According to a preferred embodiment of the preparation station, provision is made for the placement device to comprise at least one placement drive and a connecting device for a connection, preferably a detachable or non-detachable connection, to the cleaning covering, wherein the cleaning covering connected by means of the connecting device can be moved onto the cleaning main body, preferably by being unrolled from a roll, by means of the placement drive until an operating position is reached.

For example, a connecting device can be a clamp or a spool for rolling up a cleaning covering.

In an operating position of the cleaning covering, the cleaning covering is arranged on the cleaning component in such a way that the cleaning unit is operational and can be used to clean the housing. By preference, provision can be made for the cleaning covering to partly or fully cover the cleaning main body in the operating position.

A preferred embodiment variant can provide for the placement device to be provided for stretching the cleaning covering on a cleaning main body.

According to a preferred embodiment of the preparation station, provision is made for the cleaning product to be able to be applied onto and/or into the cleaning covering by means of the application device before, during and/or after a placement of the cleaning covering on the cleaning main body.

According to a preferred embodiment of the preparation station, provision is made for the application device to comprise at least one spray nozzle and/or a trough and/or a reservoir and/or an application roller and/or an application roll for applying the cleaning product to the cleaning covering.

A preferred embodiment of the preparation station provides for provision to be made for a storage container having a plurality of cleaning coverings, preferably wherein the cleaning coverings are pretreated with a cleaning product.

In a preferred embodiment variant, provision can be made for the cleaning coverings from the storage container to be prewetted with the cleaning product.

Individual cleaning coverings from the storage container can be stretched onto the cleaning unit by hand and/or automatically.

A preferred embodiment of the preparation station provides for provision to be made for a transfer device for transferring the cleaning unit between the preparation station and a medical imaging device, wherein the transfer device is drivable by hand and/or motor driven.

In a preferred embodiment variant, provision can be made for the transfer device to be at least partly automated, preferably completely automated.

A preferred embodiment of the preparation station provides for provision to be made for a displacement device, wherein the cleaning unit detachably connected to the preparation station is movable toward the medical imaging device and/or away from the medical imaging device and/or in at least one direction, preferably in at least two directions, within the medical imaging device by means of the displacement device.

A displacement device can cause the cleaning unit to move, preferably in at least partly automated fashion, particularly preferably in fully automated fashion, from a preparation position into the housing, to perform the housing cleaning there and to subsequently move back into the preparation position. Consequently, the preparation station not only enables the preparation of the cleaning unit but also enables the housing cleaning, whereby this combination of functionalities can lead to the attainment of simpler and more efficient preparation of the cleaning unit, of the housing cleaning and of the operation of the medical imaging device.

In a preferred embodiment variant, provision can be made for the displacement device to be at least partly automated, preferably be completely automated.

Statements made previously as regards automated devices and open-loop and/or closed-loop control units and sensors apply analogously to the transfer device and/or the displacement device.

According to a preferred embodiment of the preparation station, provision is made for the preparation station as a whole to be mobile, preferably movable by way of casters, or stationary.

A preferred embodiment variant can provide for the preparation station to comprise at least one securing device for securing the mobile preparation station, particularly preferably at least one brake device, or at least a securing device for securing the stationary preparation station, particularly preferably at least one belt.

According to a preferred embodiment of the preparation station, provision is made for the support structure to be dimensionally stable and/or for the cleaning component to be elastically deformable.

A higher pressure of the pressure medium and hence a greater spatial extension can be attained by way of the elastic deformability of the cleaning component, whereby the efficiency of the housing cleaning can be increased.

According to a preferred embodiment of the preparation station, provision is made for electronic components for preparing the cleaning unit to be arranged largely, preferably completely, on or in the preparation station.

In a preferred embodiment variant, provision can be made for the cleaning unit to be at least partly free, preferably completely free from metallic and/or electronic components.

In a preferred embodiment variant, provision can be made for the support structure and/or the cleaning component to contain, or consist of, at least one non-metallic material, preferably wherein the at least one non-metallic material contains, or consists of, at least one of the following materials:
natural materials, particularly preferably graphite,
polymer materials, particularly preferably elastomers, thermoplastics, thermosets, copolymers,
inorganic materials, particularly preferably ceramics or glass,
composites, particularly preferably reinforced fiber composites or laminate composites.

Electronic components and/or metallic components can adversely affect medical imaging and/or can be damaged by the magnetic field used in medical imaging. An arrangement of these components in the preparation station rather than the cleaning unit therefore leads to more reliable imaging, avoids damage and/or reduces the preparation outlay.

With the above and other objects in view there is also provided, in accordance with the invention, an arrangement having a preparation station as described and having a medical imaging device. The medical imaging device comprising a scanner unit, a patient-accommodating region at least partly surrounded by the scanner unit and a housing that surrounds the patient-accommodating region, and has a cleaning unit for cleaning the housing.

According to a preferred embodiment of the arrangement, provision is made for the preparation station to be arranged along a closed side of the scanner unit or on an open side of the scanner unit.

No opening of the housing is blocked by the arrangement along a closed side of the scanner unit, whereby the accessibility of the housing for imaging and/or cleaning is increased.

Arrangement along an open side of the scanner unit blocks an opening in the housing but, in return, the preparation station can be moved into the housing, within the housing and out of the housing for the purpose of cleaning the housing should a displacement device be present. By preference, provision can be made for the displacement device to be part of the preparation station or a displacement device of the medical imaging device, for example a scanner table.

According to a preferred embodiment of the arrangement, provision is made for the arrangement to comprise a displacement device, preferably with a scanner table, separate from the preparation station, wherein the cleaning unit can be transferred from the preparation station to the separate displacement device, preferably to the scanner table, and/or the cleaning unit can be transferred from the separate displacement device, preferably from the scanner table, to the preparation station.

In the case of a separate displacement device, preferably with a scanner table, it is advantageous for the preparation station to be arranged on an open side of the scanner unit.

In a preferred embodiment variant, provision can be made for the cleaning component to comprise a cleaning main body and a cleaning covering, wherein the cleaning covering is placed along an outer contour of the cleaning main body.

In a preferred embodiment variant, provision can be made for the cleaning component to be arranged along a contour, preferably an outer contour, of the support structure, wherein the cleaning component is deformable by filling with a pressure medium.

Here, a “cleaning component deformable by filling with a pressure medium” can be understood to mean that a cleaning component is deformable by virtue of a volume determined in part, preferably in full by the cleaning component being taken up at least in part, preferably in full by a pressure medium.

In a preferred embodiment variant, provision can be made for the cleaning component of the cleaning unit inserted into the housing to be arranged between the support structure and the housing, wherein the cleaning component, in a pressure medium-filled state, rests against the housing, preferably with contact pressure.

In a preferred embodiment variant, provision can be made for the cleaning main body to be able to be a tubular body.

In a preferred embodiment variant, provision can be made for the cleaning main body to be able to be a flat material web, preferably a membrane, wherein at least one end, preferably at least two ends, of the flat material web, preferably of the membrane, can be connected to the support structure, preferably along an outer contour of the support structure.

In a preferred embodiment variant, provision can be made for the cleaning main body to be able to be an extruded profile and/or an extruded double profile.

In a preferred embodiment variant, provision can be made for the cleaning covering to comprise a foam and/or a textile structure and/or a nonwoven material.

In a preferred embodiment variant, the cleaning covering may be a sponge and/or a nonwoven fabric.

In a preferred embodiment variant, the cleaning covering may be a squeegee lip.

In a preferred embodiment variant, provision can be made for the cleaning covering to be moistened or unmoistened.

In a preferred embodiment variant, provision can be made for a spatial extension, preferably an external diameter, of the cleaning unit
to be smaller in the event of a non-filled cleaning component than the spatial extension, preferably the external diameter, of the cleaning unit with a filled cleaning component, and/or
to be smaller in the event of a non-filled cleaning component than a spatial extension, preferably an internal diameter, of the housing, and/or
to be at least as large or larger in the event of a filled cleaning component as/than a spatial extension, preferably an internal diameter, of the housing.

In a preferred embodiment variant, provision can be made for the pressure medium to be a gas or a liquid, preferably compressed air.

A gas or a liquid as pressure medium can be accompanied by different advantages. For example, gases are compressible and can therefore better compensate pressures that are too high. Liquids are hardly compressible and therefore transmit pressures virtually without pressure loss.

Other features which are considered as characteristic for the invention are set forth in the appended claims.

Although the invention is illustrated and described herein as embodied in a method for preparing a cleaning unit by means of a preparation station, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 shows a schematic, perspective view of a medical imaging device;

FIG. 2 shows a schematic, perspective view of a preparation station;

FIGS. 3 and 4 show two schematic, perspective views of an embodiment variant of the cleaning unit;

FIGS. 5 and 6 show two schematic side views of two embodiment variants of an arrangement having a preparation station and a cleaning unit;

FIGS. 7 to 9 show three schematic side views of two embodiment variants of an arrangement having a preparation station, a cleaning unit and a medical imaging device;

FIGS. 10 and 11 show two schematic side views of a further embodiment variant of an arrangement having a preparation station, a cleaning unit and a medical imaging device; and

FIG. 12 shows a schematic plan view of an arrangement having a cleaning unit and a medical imaging device and different positions of a preparation station.

### DETAILED DESCRIPTION OF THE INVENTION

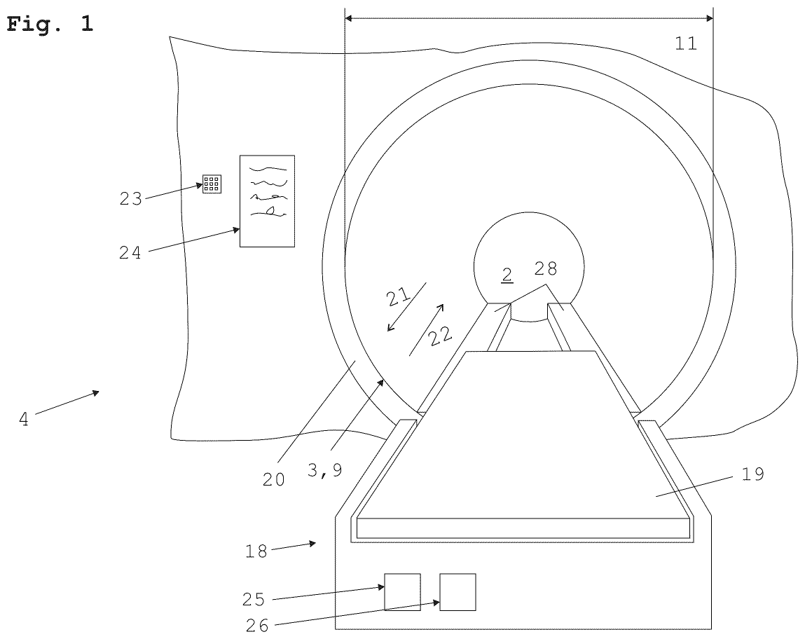

Referring now to the figures of the drawing in detail and first, in particular, to FIG. 1, which shows a schematic perspective view of a medical imaging device 4. The medical imaging device 4 has a scanner unit 20, a patient-accommodating region 2 at least partly surrounded by the scanner unit 20, and a housing 3 that surrounds the patient-accommodating region 2.

Such a medical imaging device 4 can comprise at least one cleaning unit 1, wherein the cleaning unit 1 can be movable within the housing 3.

Such a medical imaging device 4 can comprise an operating device 23 and an output device 24.

Such a medical imaging device 4 can comprise a displacement device 18. The displacement device 18 can comprise a movable scanner table 19 or patient support 19, a displacement drive 25, and an open-loop and closed-loop control unit 26.

The displacement device 18 is configured to move the scanner table 19 in at least one direction 21, 22, preferably in a first direction 21 and in a second direction 22.

The housing 3 of the illustrated device has an inner contour 9, wherein the inner contour 9 can be circular or arc-shaped, as shown in FIG. 1.

The housing 3 and/or an inner contour 9 of the housing 3 has a spatial extension 11 of the housing 3.

In order to illuminate the patient-accommodating region 2 within the housing 3, the medical imaging device 4 may comprise light strips 28 within the housing 3.

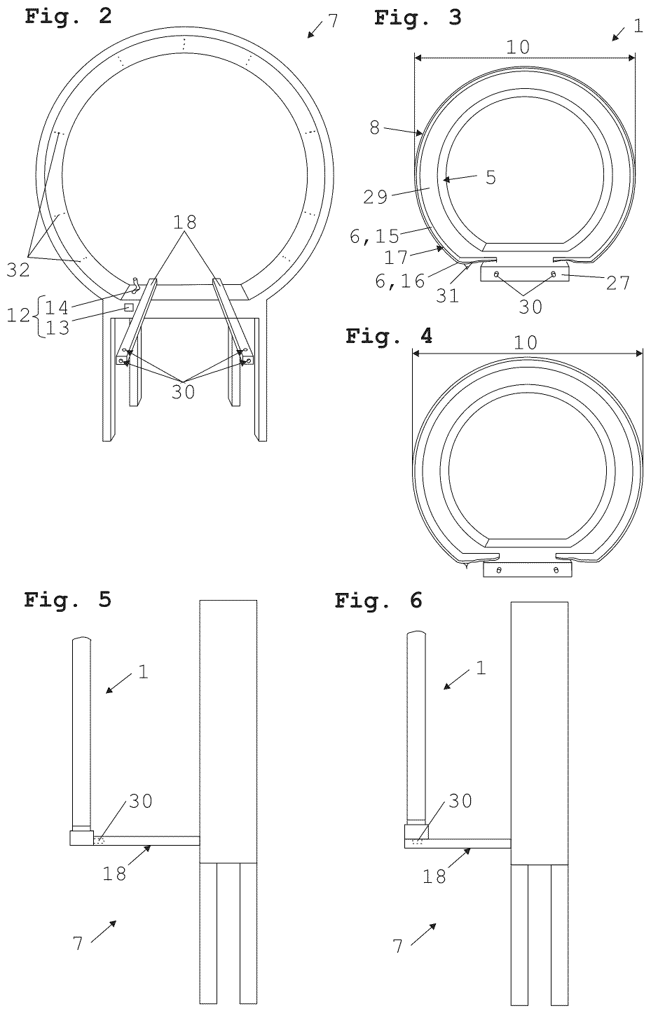

Referring now to FIG. 2, there is shown a schematic, perspective view of a preparation station 7.

The preparation station 7 for preparing a cleaning unit 1, which is provided to clean a housing 3 of a medical imaging device 4 that surrounds a patient-accommodating region 2 and the cleaning unit 1 comprising a support structure 5 and a cleaning component 6. The preparation station 7 is separate from the medical imaging device 4 and arranged in the vicinity of same. The preparation station 7 comprises at least one preparation device, by means of which the cleaning unit 1 is prepared for subsequent cleaning.

In FIG. 2, the preparation station 7 has a substantially ring-shaped, specifically an arc-shaped, upper part and a lower table, on which the upper part is arranged.

Nozzles for applying a cleaning product can be arranged within the upper part along the inner contour of the said upper part. These nozzles represent an application device 32, wherein an individual reference sign has not been provided for each nozzle in FIG. 2 for reasons of clarity. If a cleaning unit 1 is situated within the upper part or, in other words, if a cleaning unit 1 is arranged such that the cleaning unit 1 is surrounded by the preparation station 7, specifically the application device 32, then a cleaning product can be applied to the cleaning component 6 of the cleaning unit 1.

The preparation station 7 in FIG. 2 comprises a deformation device 12 for deforming the cleaning component 6, wherein the deformation device 12 comprises a deformation drive 13, a pressure medium and a connecting line 14 for connecting, preferably detachably or non-detachably connecting, the cleaning component 6 to the deformation drive 13, wherein the pressure medium can be filled into the cleaning component 6 and/or removed from the cleaning component 6 by means of the deformation drive 13, wherein the cleaning component 6 is deformable by filling with the pressure medium and/or removing the pressure medium.

For example, the deformation drive 13 can be an air compressor which conveys air from the surroundings through the connecting line 14 and hence fills the cleaning component 6 with compressed air.

The preparation station 7 in FIG. 2 comprises a displacement device 18 with two displacement rails, wherein a respective coupling device 30 for detachably connecting the cleaning unit 1 to the preparation station 7 on the front side and on the top side of the displacement rails is provided on each displacement rail. Hence, a cleaning unit 1 can be arranged on the front side and/or on the top side of the displacement rails.

FIG. 3 shows a schematic, perspective view of an embodiment variant of the cleaning unit 1 in the non-filled state.

The cleaning unit 1 can comprise a support structure 5 and a cleaning component 6.

The support structure 5 can comprise a base part 27 and a ring part 29, wherein the base part 27 can comprise at least one coupling device 30 for the detachable connection to a displacement device 18.

The position of the coupling devices 30 can be provided on the base part 27 and/or on the ring part 29. Different positions of the at least one coupling device 30 might be advantageous in the cases that the displacement device 18 for moving the cleaning unit 1 comprises a scanner table 19 and/or that the displacement device 18 is a device that is separate from the medical imaging device.

The number and positions of at least one coupling device 30 can be provided according to requirements. The illustrated embodiment variant should not be construed as limiting.

The ring part 29 of the support structure 5 in FIGS. 3 and 4 has an outer contour which describes virtually a complete circle, specifically an arc having an angle of more than 180°. Provision can be made for the support structure 5 to have an outer contour which has a complete circle or an incomplete circle, specifically an arc.

The cleaning component 6 can comprise a cleaning main body 15 and a cleaning covering 16, wherein the cleaning main body 15 might be arranged between the support structure 5, specifically the ring part 29 and/or the outer contour of the support structure 5, and the cleaning covering 16.

Provision can be made for the cleaning component 6 to extend onward beyond the arc of the ring part 29 to the base part 27. This allows both the inner side of the housing 3 and at least one side of the light strips 28, specifically the top side of the light strip 28, to be cleaned. With the aid of the embodiment variant of FIGS. 3 and 4, the cleaning component 6 also covers the gap that is found between the light strips 28 and the base part 27.

The cleaning covering 16 can have a flexible embodiment such that the cleaning covering 16 adapts to the shape of the cleaning main body 15. In FIGS. 3 and 4, the shape of an outer contour of the cleaning main body 15 substantially corresponds to the shape of the outer contour of the cleaning covering 16 arranged on the cleaning main body 15.

In FIGS. 3 and 4, the shape of an outer contour of the support structure 5 substantially corresponds to the shape of the outer contour 8 of the cleaning component 6, specifically in FIGS. 3 and 4 the outer contour of the cleaning covering 16 arranged on the cleaning component 6.

In FIGS. 3 and 4, the shape of the outer contour 8 of the cleaning component 6 is substantially circular, specifically arc shaped.

The cleaning component 6 can comprise a connector 31 in order to connect, preferably detachably or non-detachably connect, the cleaning component 6 to the connecting line 14. The connector can be sealable and can be opened and/or closed either manually or automatically.

FIG. 4 shows a schematic, perspective view of an embodiment variant of the cleaning unit 1 in the filled state. The statements made in the context of FIG. 3 also apply analogously to FIG. 4.

In FIG. 3, the cleaning component 6 contains nothing, i.e. is in a non-filled state. What is important is that the outer contour 8 of the cleaning component 6 has a spatial extension 10 that is smaller than a spatial extension 10 of the cleaning component 6 in the filled state and/or smaller than a spatial extension 11 of the housing 3. In other words, it is important that the outer contour 8 of the cleaning component 6 is not in contact with the housing in the non-filled state.

It is quite evident in FIG. 4 that the spatial extension 10 of the cleaning component 6 is greater than the spatial extension 10 of the cleaning component 6 in FIG. 3. This filled state of the cleaning component 6 can be achieved by filling the cleaning component 6 with a pressure medium by way of the deformation device 12.

At this point, it should be observed that the cleaning unit 1 can comprise at least two similar cleaning components 6 or at least two different types of cleaning components.

FIG. 5 shows a schematic side view of an embodiment variant of an arrangement having a preparation station 7 and a cleaning unit 1, wherein the cleaning unit 1 is arranged on the front side of the displacement device 18, specifically the displacement rails, and is in a non-filled state like in FIG. 3.

In FIG. 5 and all subsequent FIGS. 6 to 10, dashed lines indicate concealed construction features. For example, in FIGS. 5 and 6, the coupling devices 30 are depicted in dashed lines because they are not visible from the outside in the side view of FIGS. 5 and 6 in light of the embodiment variant in FIG. 2.

FIG. 6 shows a schematic side view of an embodiment variant of an arrangement having a preparation station 7 and a cleaning unit 1, wherein the cleaning unit 1 is arranged on the top side of the displacement device 18, specifically the displacement rails, and is in a non-filled state like in FIG. 3.

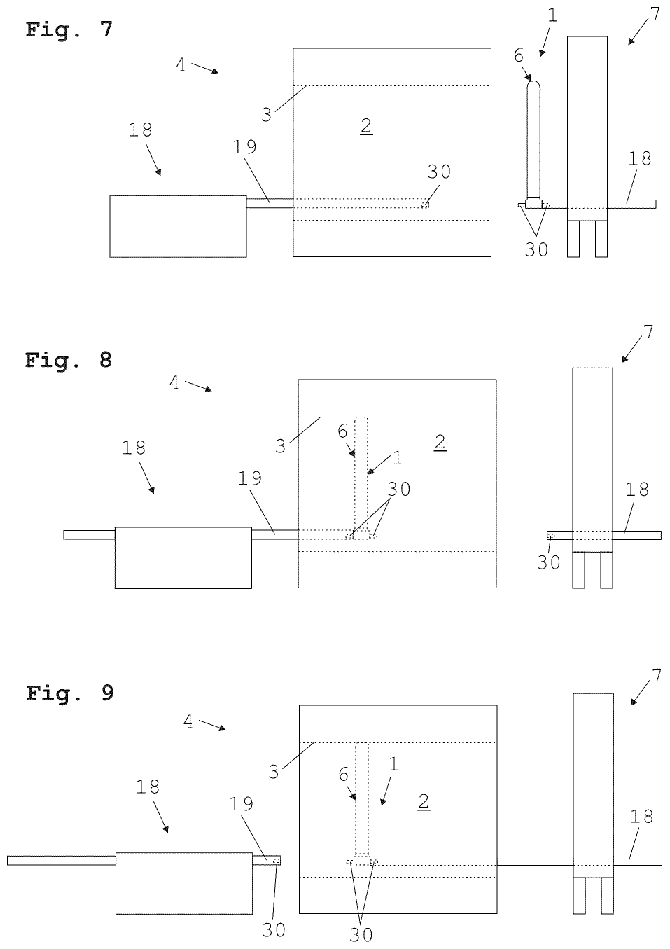

FIG. 7 shows a schematic side view of an embodiment variant of an arrangement having a preparation station 7, a cleaning unit 1, which is in a filled state like in FIG. 4, and a medical imaging device 4.

FIG. 7 shows an arrangement having a preparation station 7 according to the invention and having a medical imaging device 4, the medical imaging device 4 comprising a scanner unit 20, a patient-accommodating region 2 at least partly surrounded by the scanner unit 20 and a housing 3 that surrounds the patient-accommodating region 2, and having a cleaning unit 1 for cleaning the housing 3. In addition, a displacement device 18 that is separate from the preparation station 7 and the medical imaging device 4 is provided.

The scanner table 19 of the separate displacement device 18 is extended to such an extent next to the medical imaging device 4 in FIG. 7, left, that some of the scanner table 19 is located within the patient-accommodating region 2 within the housing 3. As illustrated in FIG. 5, the cleaning unit 1 is detachably connected to the preparation station 7.

In FIG. 7, the displacement device 18 of the preparation station 7 can move the cleaning unit 1 into the housing 3 and/or move the cleaning unit 1 out of the housing 3.

FIG. 8 shows the arrangement from FIG. 7, wherein a transfer of the cleaning unit 1 has taken place, preferably within or outside of the housing 3, from the preparation station 7 to the scanner table 19 of the separate displacement device 18. A corresponding return transfer can be provided. In this way, the cleaning unit 1 can be used to clean the housing 3 by means of the scanner table 19. For a cleaning unit 1 transfer, provision is made for coupling devices 30 on the separate displacement device 18, on the cleaning unit 1 and on the displacement device 18 of the preparation station 7.

In comparison with FIG. 7, it is quite evident on the basis of the cleaning unit 1, specifically the cleaning component 6, that the cleaning unit 1, specifically the cleaning component 6 in the filled state, is pressed against the housing 3 in FIG. 8.

FIG. 9 shows a schematic side view of a further embodiment variant of an arrangement having a preparation station 7, a cleaning unit 1, which is in a filled state like in FIG. 4, and a medical imaging device 4.

In the embodiment variant in FIG. 9, the displacement device 18 of the preparation station 7 is dimensioned such that the displacement device 18 of the preparation station 7 can also perform a displacement for cleaning the housing 3 in addition to a displacement for preparing the cleaning unit 1 since the displacement rails have a sufficiently long embodiment.

It is evident in FIG. 9 that the cleaning unit 1 can be moved from the first opening to the second opening within the housing 3 by means of the displacement device 18 of the preparation station 7, in order to clean the entire length of the housing 3.

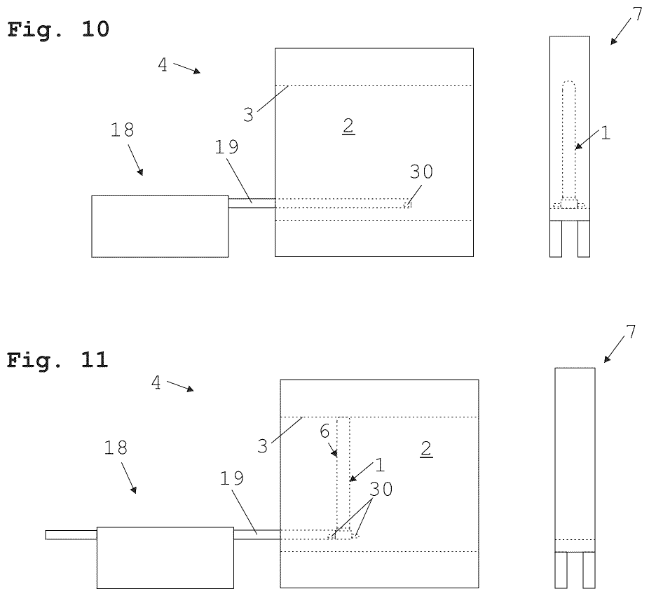

FIGS. 10 and 11 show an arrangement having a preparation station 7 according to the invention and having a medical imaging device 4, the medical imaging device 4 comprising a scanner unit 20, a patient-accommodating region 2 at least partly surrounded by the scanner unit 20 and a housing 3 that surrounds the patient-accommodating region 2, and having a cleaning unit 1 for cleaning the housing 3. In addition, a displacement device 18 that is separate from the preparation station 7 and the medical imaging device 4 is provided.

In contrast to the embodiment variants in FIGS. 7 to 9, the preparation station 7 does not comprise a dedicated displacement device 18. In this exemplary embodiment, the cleaning unit 1 is displaced only by way of the displacement device 18, and the associated scanner table 19, separate from the medical imaging device 4 and separate from the preparation station 7.

In the exemplary embodiment in FIGS. 10 and 11, the cleaning unit 1 is transferred within the preparation station 7, in which the cleaning unit is arranged, preferably parked and/or prepared, like in FIG. 10. The scanner table can be displaced up to the preparation station 7, subsequently take over the cleaning unit 1 and move out of the preparation station 7, into the housing 3 and in at least one direction 21, 22 within the housing 3.

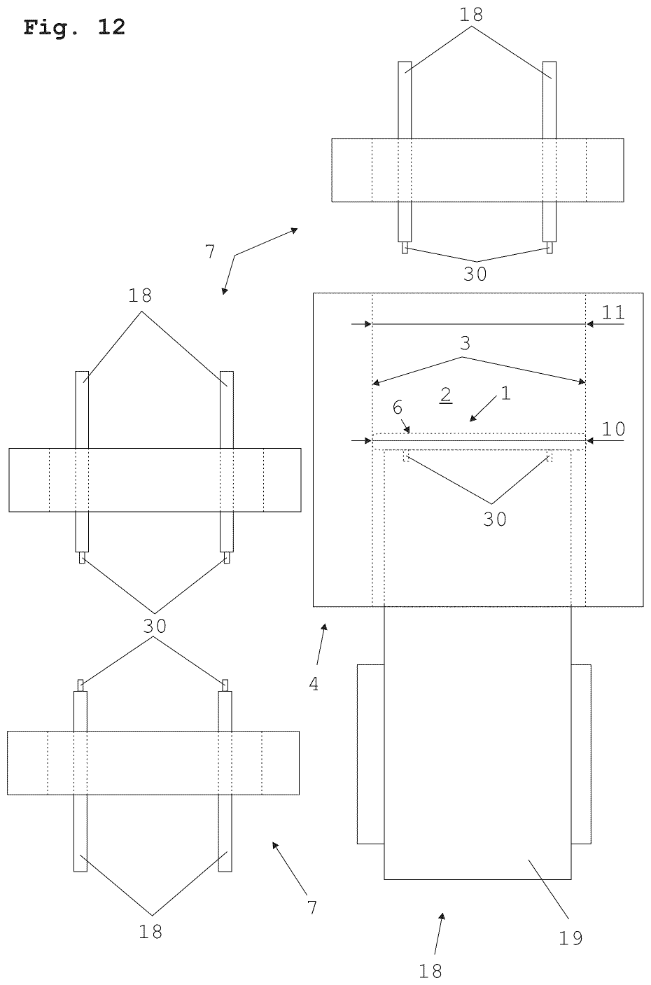

FIG. 12 shows a schematic plan view of an embodiment variant of an arrangement having a preparation station 7, a cleaning unit 1, which is in a filled state (as in FIG. 4), and a medical imaging device 4. In this case, the preparation station 7 is depicted at three different positions. It is not mandatory for three preparation stations 7 to be present; however, FIG. 12 is intended to illustrate various options for positioning the preparation station 7 by way of example. Nevertheless, more than one preparation station 7 might be provided for such an arrangement, for example advantageously if more than one cleaning unit 1 should be used.

As depicted in FIG. 12, provision can be made for the preparation station 7 to be arranged along a closed side of the medical imaging device 4, specifically of the scanner unit 20, next to it to the left in FIG. 12, or along an open side of the medical imaging device 4, specifically of the scanner unit 20, to the top in FIG. 12.

Provision can also be made for the preparation station 7 to be arranged along a closed side of the medical imaging device 4, specifically of the scanner unit 20, at the left-lower corner in FIG. 12.

If a preparation station 7 is arranged along a closed side of the medical imaging device 4, specifically the scanner unit 20, the cleaning unit can be transferred back and forth over a short path, by hand and/or by means of a transfer device, preferably in partly automated fashion, particularly preferably in fully automated fashion, between the preparation station 7 and the medical imaging device 4.

If a preparation station 7 is arranged along an open side of the medical imaging device 4, specifically the scanner unit 20, the cleaning unit can be transferred back and forth over a short path, by hand and/or by means of a transfer device, preferably in partly automated fashion, particularly preferably in fully automated fashion, and/or by means of at least one displacement device 18, preferably in partly automated fashion, particularly preferably in fully automated fashion, between the preparation station 7 and the medical imaging device 4.

In the case of an embodiment variant having at least one displacement device 18, preferably at least two displacement devices 18, as depicted in FIGS. 7 to 9, it can be advantageous for the preparation station 7 to be arranged at an opening of the medical imaging device 4, specifically of the scanner unit 20. As depicted in FIG. 12, it can be particularly preferable for the preparation station 7 to be arranged at that opening of the medical imaging device 4, specifically of the scanner unit 20, which is opposite another opening of the medical imaging device 4, specifically of the scanner unit 20, wherein the separate displacement device 18 with the scanner table 19 is arranged at the other opening.

In FIG. 12, as in FIGS. 8 and 9, it is quite evident on the basis of the cleaning unit 1, specifically the cleaning component 6, that the cleaning unit 1, specifically the cleaning component 6, is pressed against the housing 3. As a result, the spatial extension 10 of the cleaning unit 1 corresponds to the spatial extension 11 of the housing 3 because the housing 3 delimits the spatial extension 10 of the pressure medium-filled cleaning component 6, in other words the extension of the cleaning component 6 in the filled state.

The following is a summary list of reference numerals and the corresponding structure used in the above description of the invention:
1 Cleaning unit
2 Patient-accommodating region
3 Housing
4 Medical imaging device
5 Support structure
6 Cleaning component
7 Preparation station
8 Outer contour of the cleaning component
9 Inner contour of the housing
10 Spatial extension of the cleaning unit
11 Spatial extension of the housing
12 Deformation device
13 Deformation drive
14 Connecting line
15 Cleaning main body
16 Cleaning covering
17 Outer contour of the cleaning main bodies
18 Displacement device
19 Scanner table, patient table, scanner bed
20 Scanner unit
21 First direction
22 Second direction
23 Operating device
24 Output device
25 Displacement drive
26 Open-loop and closed-loop control unit of the displacement device
27 Base part
28 Light strip
29 Ring part
30 Coupling device
31 Connector
32 Application device

1. A method for preparing a cleaning unit that is configured for cleaning a housing of a medical imaging device surrounding a patient-accommodating region, the cleaning unit having a support structure and a cleaning component, the method comprising:
providing a preparation station separate from the medical imaging device and arranged in a vicinity of the medical imaging device;
moving the cleaning unit towards the preparation station; and
subsequently carrying out at least one preparation step for preparing the cleaning unit for a subsequent cleaning.

2. The method according to claim 1, comprising the following method steps:
detachably connecting the cleaning unit to the preparation station;
charging at least one of a force accumulator or an energy accumulator;
deforming the cleaning component by filling with a pressure medium or by removing the pressure medium;
placing a cleaning covering of the cleaning component on a cleaning main body of the cleaning component; and
applying a cleaning product to a cleaning covering of the cleaning component.

3. The method according to claim 2, wherein:
the cleaning unit is detachably connected to the preparation station by way of a coupling device;
the deforming step comprises inflating or deflating the cleaning component by filling with a pressure medium or by removing the pressure medium;
the placing step comprises stretching the cleaning covering of the cleaning component on the cleaning main body along an outer contour of the cleaning main body;
the cleaning product is applied to the cleaning covering before, during, and/or after a placement of the cleaning covering on the cleaning main body of the cleaning component.

4. The method according to claim 1, which comprises at least one of the following method steps:
detaching the cleaning unit that is detachably coupled to the preparation station;
transferring the cleaning unit between the medical imaging device and the preparation station;
detachably connecting the cleaning unit to the medical imaging device; and
detaching the cleaning unit from the medical imaging device.

5. A method of cleaning a housing of a medical imaging device that surrounds a patient-accommodating region, the method comprising:
providing a cleaning unit, the cleaning unit having a support structure and a cleaning component; and
providing a preparation station, that is separate from and arranged in a vicinity of the medical imaging device, the preparation station having at least one preparation device and at least one displacement device, and the cleaning unit being detachably connected to the preparation station;
carrying out at least one preparation step by the at least one preparation device to thereby prepare the cleaning unit for a subsequent cleaning; and
cleaning the housing of the medical imaging device by moving the cleaning unit within the medical imaging device in at least one direction.

6. A preparation station for preparing a cleaning unit, the cleaning unit being configured for cleaning a housing of a medical imaging device that surrounds a patient-accommodating region and the cleaning unit having a support structure and a cleaning component, the preparation station being separate from the medical imaging device and to be arranged in a vicinity thereof, the preparation station comprising:
at least one preparation device configured to prepare the cleaning unit for subsequent cleaning.

7. The preparation station according to claim 6, wherein said at least one preparation device is at least one of:
a coupling device for detachably connecting the cleaning unit to the preparation station;
a charging device for charging at least one of a force accumulator or an energy accumulator;
a deformation device for deforming the cleaning component;
a placement device for placing a cleaning covering of the cleaning component on a cleaning main body of the cleaning component; or
an application device for applying a cleaning product onto and/or into a cleaning covering of the cleaning component.

8. The preparation station according to claim 7, wherein the deformation device comprises a deformation drive, a pressure medium and a connecting line for connecting the cleaning component to the deformation drive, wherein the pressure medium is to be filled into, or removed from, the cleaning component by the deformation drive, and wherein the cleaning component configured to for deformation by filling with the pressure medium or by removing the pressure medium.

9. The preparation station according to claim 7, wherein said placement device is configured for stretching the cleaning covering on said cleaning main body, along an outer contour of said cleaning main body.

10. The preparation station according to claim 7, wherein said placement device comprises a placement drive and a connecting device for a connection to said cleaning covering, wherein said cleaning covering connected by way of said connecting device is movable onto said cleaning main body by means of the placement drive until an operating position is reached.

11. The preparation station according to claim 7, wherein said cleaning product is to be applied onto and/or into the cleaning covering by way of said application device before, during and/or after a placement of said cleaning covering on said cleaning main body.

12. The preparation station according to claim 7, wherein said application device comprises at least one element selected from the group consisting of a spray nozzle, a trough, a reservoir, an application roller, and an application roll for applying the cleaning product to said cleaning covering.

13. The preparation station according to claim 6, further comprising a storage container containing a plurality of cleaning coverings that are pretreated with a cleaning product.

14. The preparation station according to claim 6, further comprising a transfer device for transferring said cleaning unit between said preparation station and a medical imaging device, wherein said transfer device is drivable by hand and/or by a motorized drive.

15. The preparation station according to claim 6, further comprising a displacement device configured to move said cleaning unit, which is detachably connected to said preparation station, toward said medical imaging device or away from said medical imaging device and/or within said medical imaging device.

16. The preparation station according to claim 6, wherein said support structure is dimensionally stable and/or said cleaning component is elastically deformable.

17. The preparation station according to claim 6, further comprising electronic components for preparing said cleaning unit disposed on or in said preparation station.

18. An arrangement comprising a preparation station according to claim 6; and
a medical imaging device having a scanner unit, a patient-accommodating region at least partly surrounded by said scanner unit, and a housing surrounding said patient-accommodating region; and
a cleaning unit for cleaning said housing.

19. The arrangement according to claim 18, wherein said preparation station is arranged along a closed side of said scanner unit or on an open side of said scanner unit.

20. The arrangement according to claim 18, further comprising:
a displacement device, being a scanner table, separate from said preparation station, wherein said cleaning unit is to be transferred from said preparation station to said separate displacement device and/or said cleaning unit is to be transferred from said separate displacement device to said preparation station.
